(19) United States
(12) Patent Application Publication
(10) Pub. No.: US 2022/0322784 A1
(43) Pub. Date: Oct. 13, 2022

(54) SELF-RESONATING WIRELESS SENSOR SYSTEMS AND METHODS

(71) Applicant: 3M INNOVATIVE PROPERTIES COMPANY, St. Paul, MN (US)

(72) Inventors: Kristy A. Jost, Woodbury, MN (US); Caitlin A. McCandler, Berkeley, CA (US); Mark V. Riofski, Eagan, MN (US); Frank J. Kovacs, Edina, MN (US); Brian E. Brooks, St. Paul, MN (US)

(21) Appl. No.: 17/753,462

(22) PCT Filed: Sep. 3, 2020
(86) PCT No.: PCT/IB2020/058220

§ 371 Date: Mar. 3, 2022

Related U.S. Application Data
(60) Provisional application No. 62/896,697, filed on Sep. 6, 2019.

(51) Int. Cl. A43B 3/44; A41D 1/00; A61B 5/00

(52) U.S. Cl. CPC A43B 3/44; A41D 1/002; A61B 5/6807

(57) ABSTRACT

A system and method of detecting changes in an environment of an open circuit resonator configured to generate a signal when wirelessly powered by an external oscillating magnetic field, wherein the signal varies as a function of one or more environmental factors associated with the environment about the open circuit resonator. A monitoring device receives the signal from the open circuit resonator, captures data representative of the signal, compares the captured data to data previously received from the sensor to determine changes in the data, and estimates, based on the changes in the data, changes in one or more of the environmental factors.

### TECHNICAL FIELD

The present application relates generally to wireless sensors and, in particular, to self-resonating wireless sensor systems and methods.

### BACKGROUND

Open circuit resonator sensors (such as Sans Electrical Connections (SansEC) sensors) are patterns of electrically conductive material that are self-resonating. Each resonator sensor is a passive antenna that is electrically powered when exposed to external oscillating magnetic fields. When powered, the resonator sensor radiates magnetic fields with characteristics that change as a function of changes in the environment in which the resonator sensor operates.

Typically, open circuit resonator sensors are fabricated without electrical connections. In some approaches, the sensors are wirelessly powered and interrogated, eliminating the need for wiring harnesses.

### SUMMARY

This disclosure describes applications of a sensor configured to resonate at a resonant frequency when exposed to an external oscillating magnetic field. The resonant frequency varies as a function of one or more environmental factors around the sensor. In addition, parameters such as return loss and peak resistance may vary as a function of one or more environmental factors around the sensor. An interrogation module is configured to generate the external oscillating magnetic field, to receive a signal from the sensor generated in response to the external oscillating magnetic field and to determine changes in the one or more environmental factors based on the signal.

In one example approach, the signal, acquired by S11 return loss measurements, is a combination of a return loss (−dB), a frequency (Hz), and a resistance (R), all variables which can shift individually based on environmental factors. The resonant frequency may depend on the conductivity, dielectric permittivity, and the geometry of the resonator, and such parameters may be engineered to acquire a resonant frequency. In some example approaches, the open circuit resonator sensor is formed from a solid metal that is etched, printed, or otherwise applied to a surface. In some example approaches, the open circuit resonator sensor is formed from conductive yarns, wires, and fabrics. In some example approaches, the open circuit resonator sensor is incorporated into a textile (forming a textile assembly) by sewing or stitching a conductive thread into a non-conductive textile substrate, or by knitting or weaving in a conductive yarn as part of the textile substrate.

In one example, a system includes an article having an open circuit resonator sensor, wherein the sensor includes an approximately planar open circuit pattern of electrically conductive material configured to generate a signal when wirelessly powered by an external oscillating magnetic field, wherein the signal varies as a function of one or more environmental factors associated with an environment around the sensor; an interrogation module configured to generate the external oscillating magnetic field, to receive the signal generated by the sensor, and to capture data representative of the received signal; and a computing device coupled to the interrogation module, wherein the computing device comprises a memory and one or more processors coupled to the memory, wherein the memory comprises instructions that when executed by the one or more processors cause one or more of the processors to receive the captured data, compare the captured data to previously captured data, and estimate, based on the changes in the captured data, changes in one or more of the environmental factors.

In another example, a system includes an article having an open circuit resonator sensor, wherein the resonator sensor includes an approximately planar open circuit pattern of electrically conductive material configured to generate a signal when the resonator sensor is wirelessly powered by an external oscillating magnetic field, wherein the signal varies as a function of one or more environmental factors associated with an environment around the resonator sensor; an interrogation module configured to generate the external oscillating magnetic field, to receive the signal from the resonator sensor, and to capture data representative of the received signal; and a machine-learning system coupled to the interrogation module, wherein the machine-learning system applies the captured data to a trained machine-learning model to detect changes in one or more of the environmental factors.

In another example, a method of detecting changes in an environment of an open circuit resonator sensor, wherein the an open circuit resonator sensor includes an approximately planar open circuit pattern of electrically conductive material configured to generate a signal when the resonator sensor is wirelessly powered by an external oscillating magnetic field, wherein the signal varies as a function of one or more environmental factors associated with the environment around the sensor, the method comprising receiving first data representative of the signal generated by the resonator sensor at a first time; receiving second data representative of the signal generated by the resonator sensor at a second time, wherein the second time is after the first time; comparing the second data to the first data to determine changes in the second data; and estimating, based on the changes in the second data, changes in one or more of the environmental factors.

In yet another example, a method of detecting changes in an environment around a SansEC sensor, wherein the SansEC sensor includes an approximately planar open circuit pattern of electrically conductive material configured to generate a signal when wirelessly powered by an external oscillating magnetic field, where the signal varies as a function of one or more environmental factors associated with the environment about the SansEC sensor, the method comprising receiving the signal from the sensor; capturing data representative of the signal; comparing the captured data to data representative of the signal at an earlier point in time to determine changes in the data; and estimating, based on the changes in the data, changes in one or more of the environmental factors.

The techniques of this disclosure may be used to measure changes in the environment surrounding a sensor in a low cost and time-efficient manner.

### BRIEF DESCRIPTION OF DRAWINGS

FIG. 1 is a block diagram illustrating an example system for monitoring environmental conditions, in accordance with one aspect of the present disclosure.

FIGS. 2A-2D are graphs illustrating example changes in resonant frequency in an open circuit resonator sensor as a function of a change in an environmental factor, in accordance with aspects of the present disclosure.

FIG. 3 is a flowchart illustrating a method of characterizing an open circuit resonator sensor, in accordance with one aspect of the present disclosure.

FIG. 4 is a schematic and conceptual diagram illustrating features of computing device 14 of FIG. 1, in accordance with one aspect of the disclosure.

FIGS. 5A and 5B illustrate changes in return loss plots in response to changes in the distance between the interrogation module and the sensor, in accordance with one aspect of the present disclosure.

FIG. 6 illustrates the effects of including and excluding factors in the regression analysis, in accordance with aspects of this disclosure.

FIG. 7 illustrates how the response from a sensor to an interrogation module changes at a constant distance from interrogation module as the sensor rotates axially relative to the interrogation module, in accordance with one aspect of the present disclosure.

FIG. 8 is a flowchart illustrating a method of determining one or more parameters based on signals received from an open circuit resonator sensor, in accordance with one aspect of the present disclosure.

FIG. 9 is a block diagram illustrating techniques for determining when the sole of a shoe no longer provides the support needed, in accordance with one aspect of the present disclosure.

FIG. 10 is a block diagram illustrating an example approach to detecting wetness, in accordance with an aspect of this disclosure.

FIG. 11 is an illustration of an article of clothing having an inside sensor and an outside sensor, in accordance with an aspect of this disclosure.

FIG. 12 is an illustration showing a brace having an integrated open circuit resonator sensor, in accordance with an aspect of this disclosure.

FIG. 13 is an illustration showing a bandage having an integrated open circuit resonator sensor, in accordance with an aspect of this disclosure.

### DETAILED DESCRIPTION

As noted above, open circuit resonator sensors are self-resonating patterns of electrically conductive material; each sensor is a passive antenna that is electrically powered when exposed to external oscillating magnetic fields. When powered, each sensor radiates magnetic fields that change as a function of the environment in which the sensor operates. This characteristic may be used to sense changes in environmental parameters such as temperature, pressure and humidity.

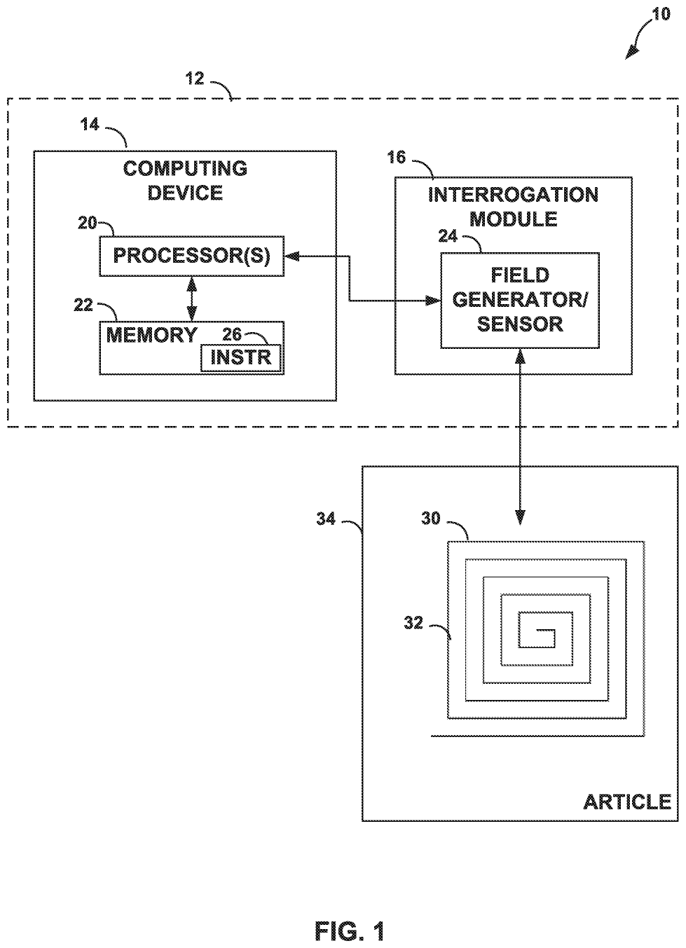

FIG. 1 is a block diagram illustrating an example system for monitoring environmental conditions, in accordance with one aspect of the present disclosure. In the example approach of FIG. 1, system 10 includes a monitoring device 12 used to query open circuit resonator sensors 30 embedded in or attached to articles 34. In the example approach of FIG. 1, monitoring device 12 includes a computing device 14 and an interrogation module 16. Computing device 14 includes one or more processors 20 connected to memory 22.

In some example approaches, interrogation module 16 includes field generator/sensor 24. In some such example approaches, interrogation module 16 generates the external oscillating magnetic field and receives a signal from the open circuit resonator sensor 30 generated in response to the external oscillating magnetic field. In one such example approach, computing device 14 is communicatively coupled to the interrogation module 16 and has a memory 22 that includes instructions 26 that when executed by the one or more processors 20 cause the one or more processors to compare data generated from the signal received from the open circuit resonator sensor 30 to data previously received from the sensor 30 to determine changes in the data and to estimate, based on the changes in the data, changes in one or more of the environmental factors.

In general, open circuit resonator sensors 30 differ from traditional antennas in that the signals generated in resonance vary based on environmental factors. It is in interpreting these differences that one can begin to take advantage of these sensors. In one example approach, sensor 30 includes an approximately planar open circuit pattern of electrically conductive material 32 configured to resonate at a resonant frequency when exposed to an external oscillating magnetic field, wherein the resonant frequency varies as a function of one or more environmental factors associated with an environment of the sensor 30. In some example approaches, sensor 30 is a planar rectangular spiral antenna such as illustrated in FIG. 1. In one such example approach, the antenna is stainless steel and is enclosed in nylon and stitched into cotton with a felt backing. The characteristic frequency of the antenna may range between 100-120 MHz depending on the environmental conditions.

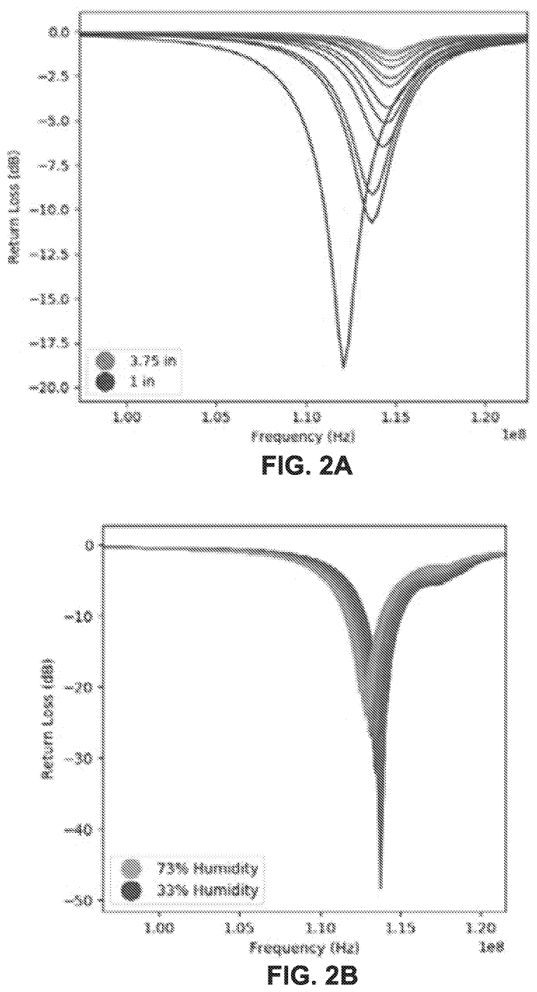

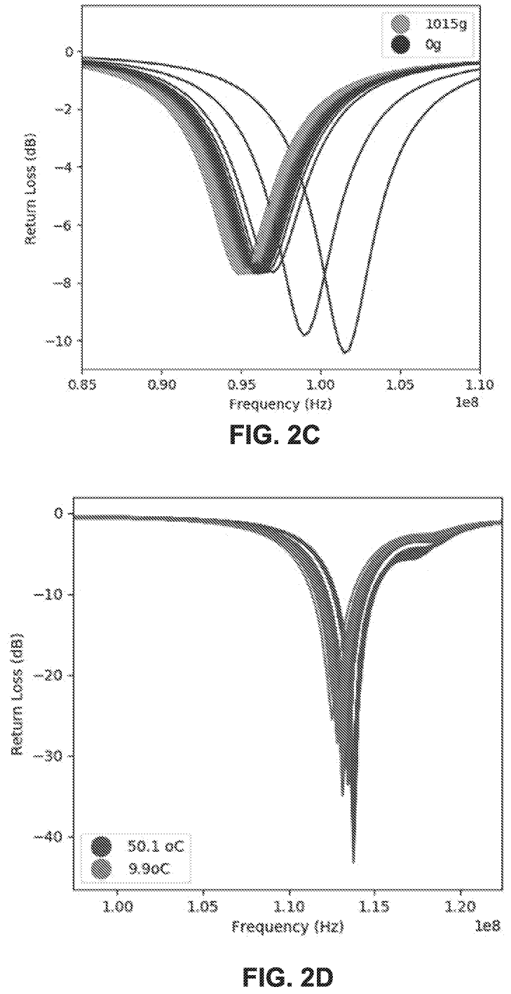

FIGS. 2A-2D are graphs illustrating example changes in resonant frequency in an open circuit resonator sensor as a function of a change in an environmental factor, in accordance with aspects of the present disclosure. In the examples shown in FIGS. 2A-2D, the sensor 30 characterized is a SansEC spiral rectangular planar antenna from Textile Instruments LLC. Signals from the antenna are correlated using specific stimuli, such as moisture, temperature, pressure and distance. In contrast to traditional antennas, signals from sensors 30 are interpreted based on how the sensor is being used.

A planar spiral antenna may be completely defined by the number of turns n, the turn width w, the turn spacing s, the outer diameter d_{out} and the inner diameter, d_{in}. The antenna characterized in FIGS. 2A-2D has 6 turns, a turn width of 0.5 mm, a turn spacing of 0.75 cm, an outer diameter of 8 cm, and an inner diameter of 0.75 cm. The thickness of the antenna has only a small effect on its properties. The fill ratio, p, of the antenna is defined as:

$$ρ = \frac{{d}_{out} - {d}_{in}}{{d}_{out} + {d}_{in}}$$

For the characterized antenna with a d_in of 0.75 cm and d_out of 8 cm, the fill ratio, p equals 0.83.

As noted above, sensor 30 is a passive sensor, meaning it is an open circuit that radiates via induction when exposed to external oscillating magnetic fields. The antenna absorbs energy at a certain frequency, producing a signal that will change slightly based on parameters such as temperature, humidity, applied pressure, and the distance and angle between sensor 30 and interrogation module 16. In the example shown in FIG. 1, the spiral antenna of sensor 30 is inherently a capacitor because of the arrangement of its wires in parallel with a dielectric material (in this example, the dielectric material is the felt backing material). The capacitance depends on the permeability of the dielectric material. The inductance of sensor 30 is also coupled with the inductance of the dielectric material.

FIGS. 2A-2D illustrate the response of a sensor 30 to stimulation by interrogation module 16 under different environmental conditions. In the example shown in FIGS. 2A-2D, sensor 30 is a planar rectangular spiral antenna (such as shown in FIG. 1). As noted above, the antenna is a passive sensor, an open circuit that radiates via induction when wirelessly powered by external oscillating magnetic fields. The antenna absorbs energy at a certain frequency, producing a signal that will change slightly based on the following parameters: temperature, humidity, applied pressure, and distance and angle between receiver and transmitter.

In one example approach, the data from the characterization is used to train a machine learning algorithm to determine changes in the environment based only on the signal of an antenna. In one example approach, the trained machine learning algorithm may be used, for instance, in textiles to predict the wearer's comfort level given the environmental conditions.

FIG. 2A illustrates changes in the signal as the separation between interrogation module 16 and sensor 30 goes from 1 inch to 3.75 inches. Notice how the return loss of the signal in decibels goes from approximately −18 dB to approximately 0 as the distance between interrogation module 16 and sensor 30 moves from 1 inch to 3.75 inches.

FIG. 2B illustrates changes in the return signal in response to changes in humidity. In the example shown in FIG. 2B, the return loss of the signal in decibels goes from approximately −48 dB to approximately −17 dB as humidity increases from 33% to 73%. FIG. 2C illustrates changes in the return signal in response to changes in pressure on sensor 30. In the example shown in FIG. 2C, the return loss of the signal in decibels goes from approximately −10 dB to approximately −8 dB as pressure increases from 0 g to 1015 g. In the example shown in FIG. 2C, the transition in return loss is more abrupt than that shown in FIGS. 2A and 2B, and the change in frequency is more pronounced. In the example shown in FIG. 2C, the frequency is lower than the examples shown in FIGS. 2A, 2B and 2D because the reader of interrogation module 16 was directly on the sensor, not 1 inch away. Generally, as the sensor gets closer to the reader, the frequency shifts downward.

FIG. 2D illustrates changes in the return signal in response to changes in temperature. In the example shown in FIG. 2D, the return loss of the signal in decibels goes from approximately −42 dB to approximately −17 dB as temperature decreases from approximately 50° C. to 10° C.

Although in FIGS. 2A-D the only environmental parameters displayed are limited to temperature, humidity, applied pressure, and the distance from sensor 30 to interrogation module 16, other factors cause changes in the signal returned by sensor 30. For instance, the angle between the interrogation module 16 and the plane of the sensor 30 leads to changes in return loss. In one example approach, as the interrogation module moves off a line orthogonal to the plane of sensor 30, return loss acts as shown for increasing distance in FIG. 2A. In addition, there is a clear decrease in return loss and an increase in return loss frequency with increased bending of sensor 30.

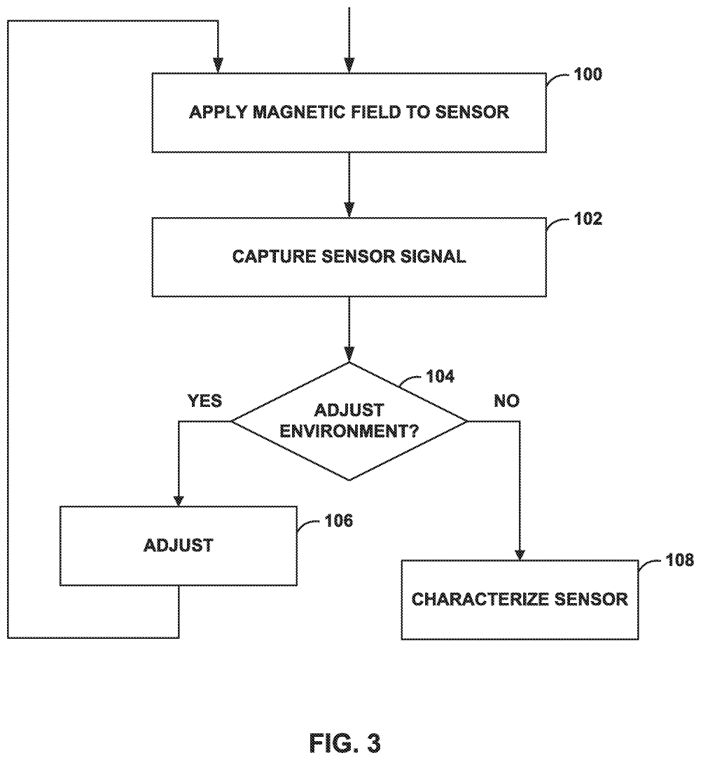

FIG. 3 is a flowchart illustrating a method of characterizing an open circuit resonator sensor, in accordance with one aspect of the present disclosure. In the example shown in FIG. 3, interrogation module 16 generates a magnetic field in the vicinity of sensor 30 (100). As noted above, sensor 30 is a passive sensor, an open circuit that radiates via induction when exposed to external oscillating magnetic fields. Sensor 30 absorbs energy at a certain frequency, producing a signal that changes as a function of changes in the environment in which sensor 30 operates and as a function of the distance and angle between sensor 30 and a transmitter in interrogation module 16. The signal from sensor 30 is captured (102). A check is made to determine if sensor signals were captured at the desired number of test points (104). If not, one or more changes are made in the sensor test environment (such as changes in the temperature at the sensor, the humidity at the sensor, the pressure on the sensor, the distance from the interrogation module 16 to the sensor 30 and the angle between the interrogation module 16 and the plane of the sensor 30 (106)) and the process is repeated (100). If sensor signals were, however, captured at the desired number of test points, computing device 14 characterizes sensor 30 based on the captured sensor signals (108). In one example approach, computing device 14 trains a machine learning algorithm with data from the captured sensor signals to predict fluctuations in the environment in which sensor 30 operates based on the signal received from sensor 30.

The effects of humidity and temperature were initially tested with two sweeps over first humidity at a constant temperature and then temperature at a constant humidity for each distance between the antenna and reader. The humidity sweep ranged between 20% and 85% humidity at a constant temperature of 25° C. while the temperature sweep ranged between 0° C. and 50° C. at a constant 50% humidity. Other approaches are contemplated. In one example approach, for instance, an environmental chamber is programmed to have 16 target set points, one for each combination of four temperature settings and four humidity settings. In one such example approach, the temperature settings range from −10° C. to 50° C. in intervals of 20° C. The humidity settings range from 40% to 70% humidity in intervals of 10% humidity. The environment is held constant for one hour at each target set point. In one such example approach, the chamber is set to the lowest temperature and the lowest percent humidity and temperature and humidity are increased over the course of the test. Return loss and resistance spectra are collected automatically every minute.

As noted above, in one example approach, computing device 14 trains a machine learning algorithm with data from the captured sensor signals to predict fluctuations in the environment in which sensor 30 operates based on the signal received from sensor 30. In one such example approach, computing device 14 implements a machine learning system used to train a machine learning algorithm. In general, each machine learning system is based on at least one model. The model may be a regression model based on techniques such as, for example, support vector regression, random forest regression, linear regression, ridge regression, logistic regression, Lasso, or nearest neighbor regression. Or the model may be a classification model based on techniques such as, for example, support vector machines, decision tree and random forest, linear discriminant analysis, neural networks, nearest neighbor classifier, stochastic gradient descent classifier, gaussian process classification, or naïve bayes. Both types of models rely on the use of labeled data sets to train the model. In one example approach, each data set represents measurements of the captured sensor signals at selected values of one or more parameters. Each data set is labeled with the selected values. In one example approach, neural web software (such as 3M Neural Network software available from 3M Company of St. Paul, Minn.) is used to create a neural network model. In one such example approach, the neural web software may be used to train a prediction based on collected data, and then evaluated for accuracy by checking the predicted responses to changes in sensor environment against the actual values.

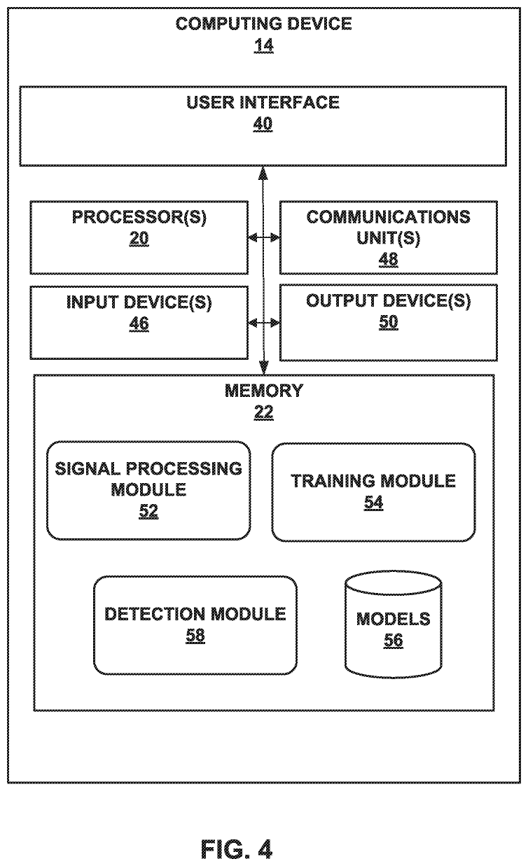

FIG. 4 is a schematic and conceptual diagram illustrating features of computing device 14 of FIG. 1, in accordance with one aspect of the disclosure. In one example approach, computing device 14 includes one or more processors 20, a memory 22, a user interface 40, one or more input devices 46, one or more communications units 48 and one or more output devices 50. User interface 40 may include a display, a graphical user interface (GUI), a keyboard, a touchscreen, a speaker, a microphone, or the like. The one or more processors 20 of computing device 14 are configured to implement functionality, process instructions, or both for execution within computing device 14. For example, processors 20 may be capable of processing instructions stored within memory 22, such as instructions for applying a trained machine-learning system to a data set to determine one or more parameters that lead to changes in return loss or in the frequency of peak resistance in sensor 30. Examples of one or more processors 20 may include any one or more of a microprocessor, a controller, a digital signal processor (DSP), an application specific integrated circuit (ASIC), a field-programmable gate array (FPGA), or equivalent discrete or integrated logic circuitry.

In some such cases, computing device 14 may include one or more input devices 46, such as, for example, a keyboard, a keypad, a touch screen, a smartphone or the like. A user may be able to indicate, using the one or more input devices, that he or she wants to detect or quantify changes experienced by sensor 30. For example, a user may be able to check off, select, or otherwise indicate using a touch screen of monitoring device 12 or another input device that he or she wants to detect or quantify changes experienced by sensor 30. In some example approaches, user interface 40 includes one or more of the input devices 46.

In some examples, computing device 14 may utilize one or more communications units 48 to communicate with one or more external devices, such as via one or more wired or wireless networks. Communications units 48 may include a network interface card, such as an Ethernet card, an optical transceiver, a radio frequency transceiver, or any other type of device configured to send and receive information. Communications units 48 may also include Wi-Fi radios or a Universal Serial Bus (USB) interface.

In some examples, one or more output devices 50 of computing device 14 may be configured to provide output to a user using, for example, audio, video or tactile media. For example, output devices 50 may include a display of user interface 40, a sound card, a video graphics adapter card, or any other type of device for converting a signal into an appropriate form understandable to humans or machines, such as a signal associated with information pertaining to a status, outcome, or other aspect of one or more data sets resulting from interrogation of one or more sensors 30 by interrogation device 16. In some example approaches, user interface 40 includes one or more of the output devices 50.

Memory 22 of computing device 14 may be configured to store information within computing device 14 during operation. In some examples, memory 22 may include a computer-readable storage medium or computer-readable storage device. Memory 22 may include temporary-use memory, meaning that a primary purpose of one or more components of memory 22 may not necessarily be long-term storage. Memory 22 may include a volatile memory, meaning memory 22 does not maintain stored contents when power is not provided thereto. Examples of volatile memories include random access memories (RAM), dynamic random-access memories (DRAM), static random-access memories (SRAM), and other forms of volatile memories known in the art. In some examples, memory 22 may be used to store program instructions for execution by processors 20, such as instructions for applying a trained machine-learning system to a data set received from interrogation module 16 via one or more communications units 48. Memory 22 may, in some examples, be used by software or applications running on computing device 14 to temporarily store information during program execution.

In one example approach, memory 22 includes information that may be used to implement functionality, process instructions, or both for execution within computing device 14. In one such example approach, memory 22 includes a signal processing module 52 that when accessed by one or more of the processors 20 may be used to implement signal processing functionality within computing device 14. The signal processing functionality may be used to receive data from interrogation module 16 representing measurements of signals received from sensor 30 in response to a magnetic field. In some such example approaches, the signal processing functionality includes functionality used to improve the quality of the data received from interrogation module 16.

In one example approach, memory 22 includes a training module 54 and a detection module 58. In one such example approach, one or more of the processors 20 access training module 54 to configure computing device 14 to train one or more machine-learning models. In some such example approaches, trained models are stored in models store 56. In one example approach, one or more of the processors 20 access detection module 58 to configure computing device 14 to apply one or more of the trained machine-learning models stored in models store 56 to signals captured from sensor 30.

In some examples, memory 22 may include non-volatile storage elements. Examples of such non-volatile storage elements include magnetic hard discs, optical discs, floppy discs, flash memories, or forms of electrically programmable memories (EPROM) or electrically erasable and programmable (EEPROM) memories. In one such example approach, signal processing module 52 may be configured to analyze data received from sensor 30, such as a data set captured by interrogation module 16 that includes measurements of the signal generated by sensor 30 in response to interrogation by interrogation module 16.

Computing device 14 may also include additional components that, for clarity, are not shown in FIG. 4. For example, computing device 14 may include a power supply to provide power to the components of computing device 14. Similarly, the components of computing device 14 shown in FIG. 4 may not be necessary in every example of computing device 14.

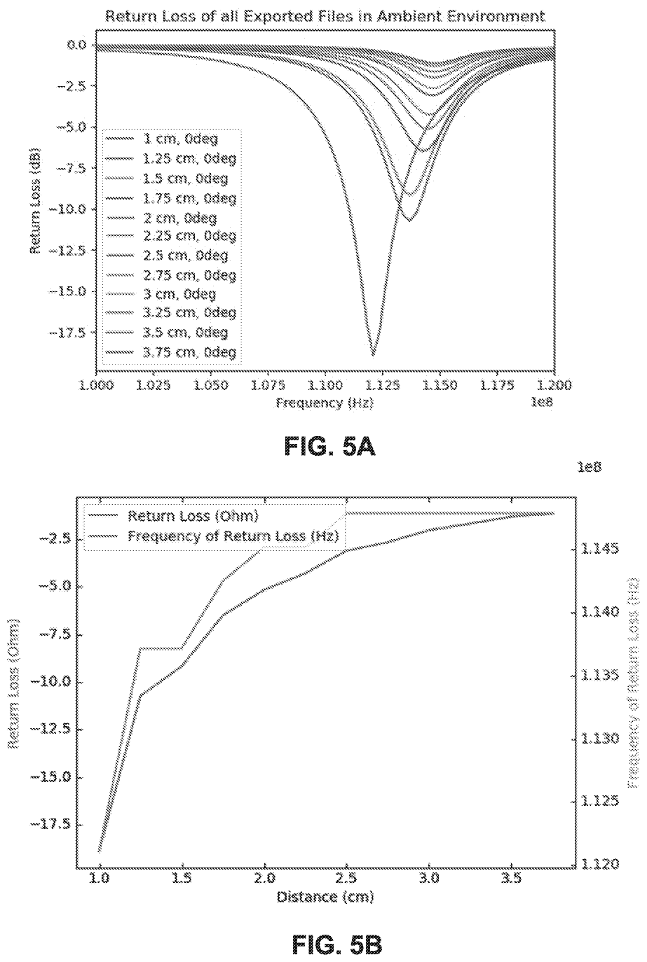

FIGS. 5A and 5B illustrate changes in return loss plots in response to changes in the distance between the interrogation module and the sensor, in accordance with one aspect of the present disclosure. In one example approach, six pieces of information are extracted from each captured sensor signal: the maximum magnitude, frequency and FWHM (Full Width at Half Maximum) of the return loss peak and the maximum magnitude, frequency and FWHM of the resistance peak. The environmental parameters for each captured sensor signal may be determined based on the respective time stamps of an environmental chamber log and the time of the signal reading. In one example approach, the data from each sensor response signal is combined in a master data set. In one example approach, data may be clustered by associating all points with the same temperature or humidity to visualize trends with variable environmental conditions. In one example approach, all data processing is performed in Python.

In one example approach, a regression model and neural network model are trained with the data collected from sensor 30 with the sensor response as an input and environmental conditions as an output. As noted above, in some example approaches, the antenna signal input includes six data points: the magnitude of return loss peak, the frequency of the return loss peak, the FWHM of return loss peak, the magnitude of the resistance peak, the frequency of peak resistance, and the FWHM of resistance peak. In one example approach, both the neural network model and the regression model predict temperature, humidity, and distance based on the signal received from sensor 30 in response to stimulation by interrogation module 16. The two models may be compared for their accuracy.

In one example approach, the model fits coefficients to the six input variables along with the squares of the input variables and the multiples of the input variables. In one example approach, a miniVNA Antenna Network Analyzer from mini Radio Solutions is used to conduct all tests on the antenna. The miniVNA has a range of up to 90 dB in transmission and 50 dB in reflection and has a frequency range of 100 kHz to 200 MHz with a step size of 1 Hz. In one such example approach, vnaJ.3.1 software may be used to collect the signal from the analyzer while a Thermotron machine may be used for environmental control.

The Textile Instruments LLC SansEC sensor 30 tested showed variations in pressure response as a function of the distance between the interrogation module 16 and the sensor 30. A sharp decrease in return loss magnitude is observed at each distance with very low weight addition. Following the initial addition of weight, the return loss magnitude increases linearly to a much smaller degree. The weight of the testing apparatus was around 25 g for each distance, so applying less than 25 g was difficult and only a few data points were collected in the range 0-25 g. The maximum point in each line occurs at the weight of the apparatus, or 25 g. Similarly, there is a sharp decrease in return loss frequency with the addition of a small amount of weight. The return loss frequency continues to decrease slightly with additional weight in an exponential trend. These results indicate the sensor's aptitude to sense small changes in pressure if the sensor is initially in a zero-pressure state. Even if the return loss magnitude were to increase to the zero-pressure state from an extremely large amount of pressure, the return loss frequency would only indicate that there is pressure applied as its trend only decreases. The frequency of peak resistance, however, stays fairly constant at each pressure, but increases the farther the interrogation module 16 is from sensor 30.

The Textile Instruments LLC SansEC sensor 30 also was tested with a variety of temperatures and a fixed humidity of approximately 50% at various distances. Return loss frequency shows good clustering at various distances, but does not consistently increase or decrease with increasing distance. A possible reason for this could be that placing the interrogator on the surface of the antenna limits the extent of induction in the antenna as the interrogator has a smaller area than the antenna. The trends in temperature may also be affected by the difficulty of achieving a stable humidity throughout the tests.

In addition, the Textile Instruments LLC SansEC sensor 30 was tested over a range of humidity settings and distances but with a fixed temperature of approximately 25° C. The tests showed that return loss decreases with increasing humidity and increasing distance between the antenna and the module 16. There appeared to be a lag in reaching the equilibrium state in the antenna at a particular humidity as a consequence of changing the humidity very rapidly over the test. As in the test of the effect of temperature, there appears to be clustering of the return loss frequency at various distances, but no consistent increase or decrease in return loss frequency with increasing distance. This effect is also apparent in the frequency of peak resistance.

As noted above, neural web software (such as Neural Network software available from 3M Company of St. Paul, Minn.) may be used to create a neural network model. In one such example approach, the neural web software may be used to train a prediction based on collected data, and then evaluated for accuracy by checking the predicted responses to changes in sensor environment against the actual values. In one example approach, neural network software developed by 3M was used to create a prediction model with data from the first round of testing in order to establish that there is predictive power in the spectra of signals generated by sensor 30. In a first round of data the experiments consisted of readings taken at three distances with temperatures ranging from 0-50° C. and humidity ranging from 20-80% humidity. The neural network model displayed the following characteristics:

Neural Network Model Results

| Variable | R² | Accuracy Range | |
|---|---|---|---|
| Distance | .991 | 1 | mm |
| Temperature | .463 | 15° | C. |

| | | |
|---|---|---|
| Humidity | .590 | 10% |

In some example approaches, classification models other than neural networks are trained based on their associated classification modeling protocols.

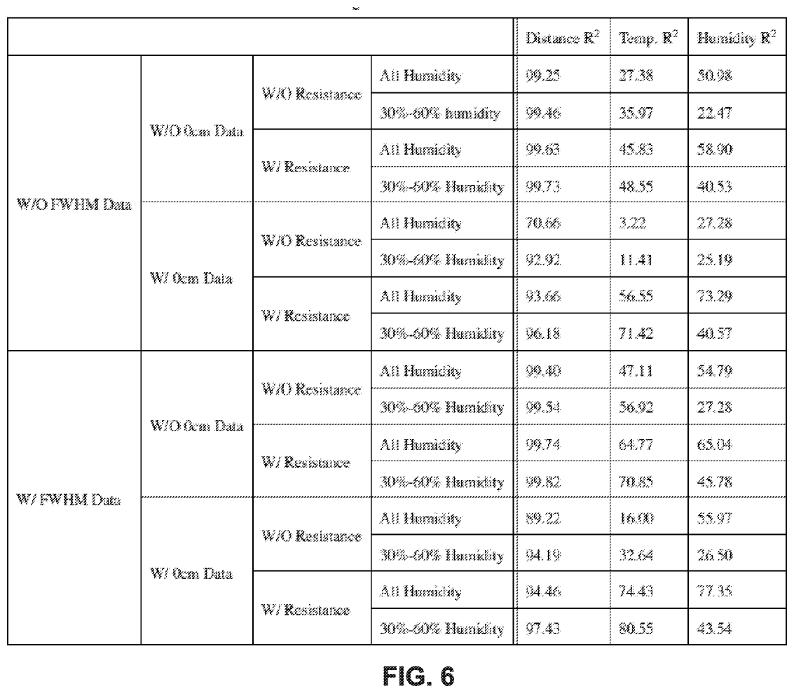

FIG. 6 illustrates the effects of including and excluding factors in the regression analysis, in accordance with aspects of this disclosure. In one example approach, Minitab software (available from Minitab, LLC of State College, Pa.) is used to create a regression model that predicts the temperature, humidity, and distance between the sensor 30 and interrogation module 16 given a signal response from sensor 30. In one example approach, regression model software evaluates the deviation of the actual data from the prediction equation by the R² of the fit. The regression model may also be trained based on other regression modeling protocols.

Appropriate regression models may be a matter of trial and error. For example, regression models may be considered that include/exclude selected data and include/exclude selected input variables, and the quality of fit for each model may be considered. There is a risk of over-fitting the model by allowing for dependencies on variables that do not have an effect on the overall system but improve the fit on the set of training data. In one example approach, input variables relating to resistance peak are included and then excluded from the model and the fits are compared. In another example approach, input variables of the FWHM of both the resistance and return loss peak are included and then excluded in the model and the fits are compared. In one example approach, data corresponding to a distance of 0 cm was included and excluded and data corresponding to humidity occurring outside the range 30%-60% was included and excluded.

By definition, allowing the regression to use more input variables will always improve the quality of fit, but this improved quality could be the result of over-fitting. From FIG. 6 and the data above, the following conclusions were determined:
1) If the model only considers 30-60% humidity, humidity sensing suffers but temperature sensing improves.
2) If the model only considers the data apart from 0 cm, its distance sensing improves but there is an ambiguous effect on temperature and humidity sensing.
3) If the model includes FWHM inputs, temperature sensing increases much more than humidity sensing.
4) If the model includes resistance inputs, the temperature sensing increases more than humidity sensing for most datasets.
   In one example, the regression model comprises:

D(mm)=69−0.000001RLF−0.656RL+0.000193R+0.000207RL

T(° C.)=−149597−0.000332RLF−62.7RL+0.00758RF+0.0078R−0.0085RL-0.000037RL*R

H(%)=−36923+0.000035RLF+36.1RL+0.00164RF−0.0605R+0.0231RL*RL+0.000044RL*R

where RLF is return loss frequency, RL is return loss, R is resistance and RF is the frequency at which peak resistance occurs. In the example regression model, the distance prediction had an R² of 0.9963, the temperature prediction had an R² of 0.4583, and the humidity prediction had an R² of 0.5890, as shown below.

Regression Model Results

| Variable | R² | Dependencies |
|---|---|---|
| Distance | .9963 | RLF, RL, R, RL*RL |
| Temperature | .4583 | RLF, RL, RF, R, RL*RL, RL*R |
| Humidity | .5890 | RLF, RL, RF, R, RL*RL, RL*R |

The effect of changes in the distance between sensor 30 and interrogation module 16, in the axial rotation of sensor 30 with respect to interrogation device 16, in bending of sensor 30, in pressure on sensor 30, and in temperature and humidity in the vicinity of sensor 30 on the signal received from sensor 30 were analyzed. As noted above, the effects of distance in one such experiment are shown in FIGS. 2A, 5A and 5B. The effects of humidity in one such experiment are shown in FIG. 2B. The effects of pressure in one such experiment are shown in FIG. 2C, while the effects of temperature in one such experiment are shown in FIG. 2D. As described above, a regression model and a neural network model were trained with the data collected and the two models were compared.

Other aspects of antenna performance may be impacted by changes occurring around sensor 30. Changes in the spacing between the interrogation module 16 and sensor 30, for instance, may have many effects, with, as noted in the discussion of FIGS. 2A, 5A and 5B, the most apparent effects being changes in the magnitude of return loss and changes in the frequency of return loss, as shown in FIGS. 2A, 5A and 5B. In the example shown in FIG. 5A, return loss was captured as sensor 30 was raised above the interrogation module 16 on a parallel plane. In one such example approach, the tests were conducted in a humidity-controlled room in which the humidity varied between 67% and 70% and the temperature varied between 73.8° C. and 74.2° C. FIG. 5A illustrates return loss plots of antenna spectra while FIG. 5B illustrates return loss and frequency at the minimum peaks at different distances between sensor 30 and interrogation module 16 for a sequence of such tests.

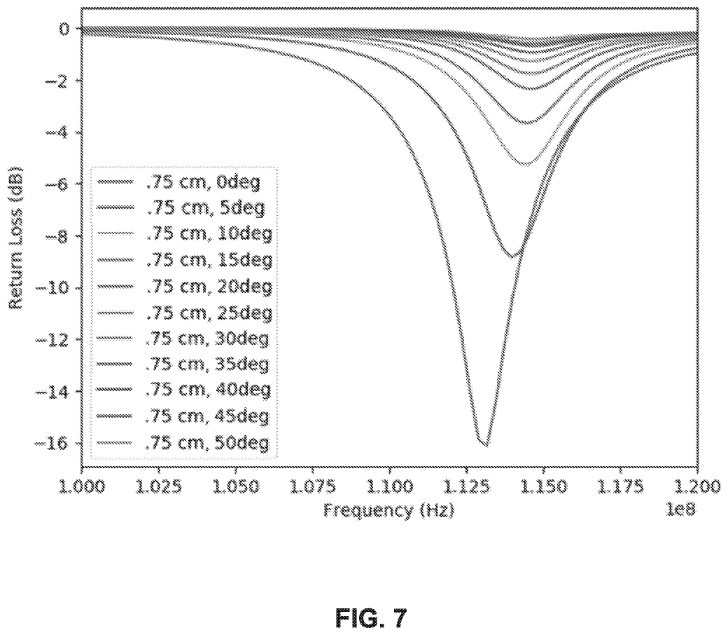

Other factors may also lead to changes in the magnitude of return loss and changes in the frequency of return loss. FIG. 7, for instance, illustrates how the response from sensor 30 to interrogation module 16 changes at a constant distance from interrogation module 16 as the sensor rotates axially relative to interrogation module 16, in accordance with one aspect of the present disclosure. In the discussion of distance above in the context of FIGS. 2A, 5A and 5B, axial rotation of sensor 30 relative to interrogation module 16 was not considered in the prediction model. Axial rotation was instead kept constant. In the example approach illustrated in FIG. 7, two tests were conducted in which the response was collected over a range of angles rotated about the midline axis and corner axis. FIG. 7 illustrates return loss at different rotations. In one example approach, a test of midline rotation had a starting distance of 2.75 cm and the rotation rotated over a range of 0-30 degrees. The test of corner rotation had a starting distance of 0.5 cm and rotated over a range of 0-50 degrees away from the sensor about the axis of the left edge of the antenna. By the nature of the rotations, only one of the orientations, at a rotation of 25 degrees, is the same in each test. In this example approach, the return loss peak point at the 25-degree rotation is −1.25 for the mid-rotation and −1.75 for the corner-rotation. The frequencies of these peaks are 1.1425 and 1.145 respectively. The temperature and humidity in the room were 74.2° F. and 67% for the midline-rotation and 74.5° F. and 70% for the corner-rotation. The deviation in temperature and humidity may correspond to the difference in return loss over the two measurements.

In one example approach, rotation and distance may be able to be combined into one factor. For instance, distance and rotation measurements taken in a humidity and temperature-controlled room were found to have aligned spectra at two measurements: (0.75 cm, 10) and (2.0 cm, 0).

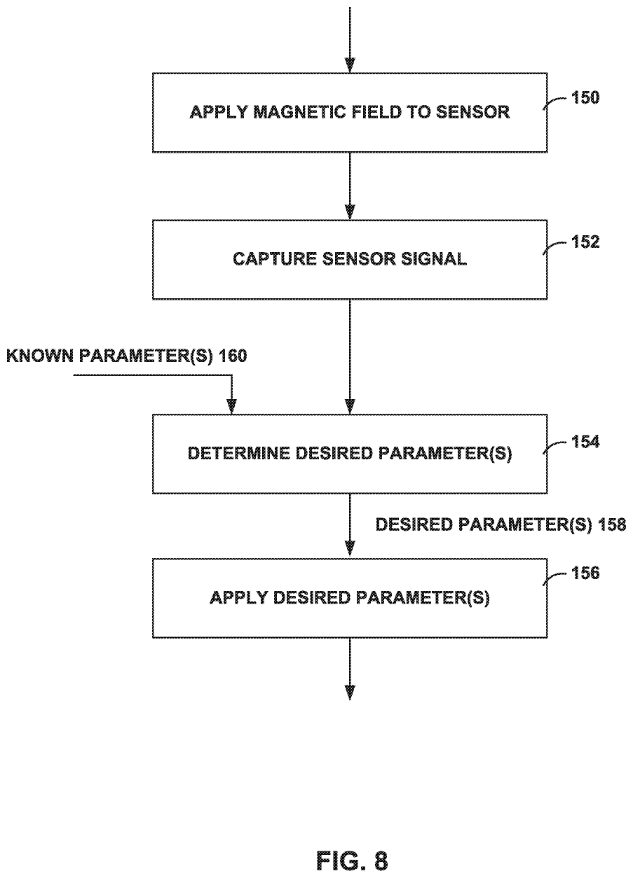

Once sensor 30 has been characterized, and the appropriate model selected, the model may be used to predict changes occurring around or to sensor 30. FIG. 8 is a flowchart illustrating a method of determining one or more parameters based on signals received from a SansEC sensor 30, in accordance with one aspect of the present disclosure. In the example shown in FIG. 8, interrogation module 16 generates a magnetic field in the vicinity of sensor 30 (150). As noted above, sensor 30 absorbs energy at a certain frequency, producing a signal that changes as a function of changes in the environment in which sensor 30 operates and as a function of the distance and angle between sensor 30 and a transmitter in interrogation module 16. The resonance signal from sensor 30 is captured (152). The captured sensor signal is then used to calculate a desired parameter (154). In one example approach, computing device 14 applies the trained machine learning algorithm described in the discussion of FIGS. 3, 4 and 5A-5B to data representing the captured sensor signal to calculate one or more such parameters. As noted above, the captured signal may be a function of one or more of temperature at the sensor, humidity at the sensor, pressure on the sensor, bending of the sensor, axial rotation, distance from the interrogation module 16 to the sensor 30 and an angle between the interrogation module 16 and the plane of the sensor 30. In one example approach, computing device 14 applies the trained machine learning algorithm described in the discussion of FIG. 3 to data representing the captured sensor signal to calculate one or more such parameters (desired parameters 158). In one such example approach, data received from external sources (known parameters 160) is used to calculate desired parameters 158. For instance, humidity and temperature readings from external sources may be used in conjunction with the trained machine learning algorithm to determine distance from the interrogation module 16 to sensor 30. The more parameters known, the more accurate the prediction.

In one such example approach, computing device 14 applies the trained machine learning algorithm described above to data representing the captured sensor signal and to data representing known parameters influencing sensor 30 to calculate the one or more desired parameters. In one example approach, the calculated parameters are used within an application to derive other parameters (156). For instance, a detected change in a parameter such as temperature or humidity may be used to determine if an environment should be heated or cooled.

SansEC sensor 30 may be used in a number of applications. For instance, sensor 30 may be used to detect wear in running shoes, to detect the presence or absence of water, in a garment to detect loss of heat, in bed linens to help a sleeper maintain a comfortable temperature, in a brace to determine if the brace is too loose or too tight, as a garden bed moisture sensor, or in a bandage to detect when a dressing is becoming too moist. In some example approaches, the electrically conductive material in sensor 30 includes one or more of a printed pattern of conductive material, a wire, a conductive yarn, a conductive fiber, and conductively coated textiles. In some such example approaches, the pattern of electrically conductive material is woven into article 34.

In some example approaches, the resonant frequency is a function of one or more of temperature at the sensor, humidity at the sensor, pressure on the sensor, the degree by which sensor 30 is bent, the distance from the interrogation module 16 to the sensor 30, axial rotation of sensor 30 relative to interrogation module 16 and an angle between the interrogation module 16 and the plane of the sensor 30. Example approaches for using sensors 30 to detect changes that affect sensor 30 are discussed next.

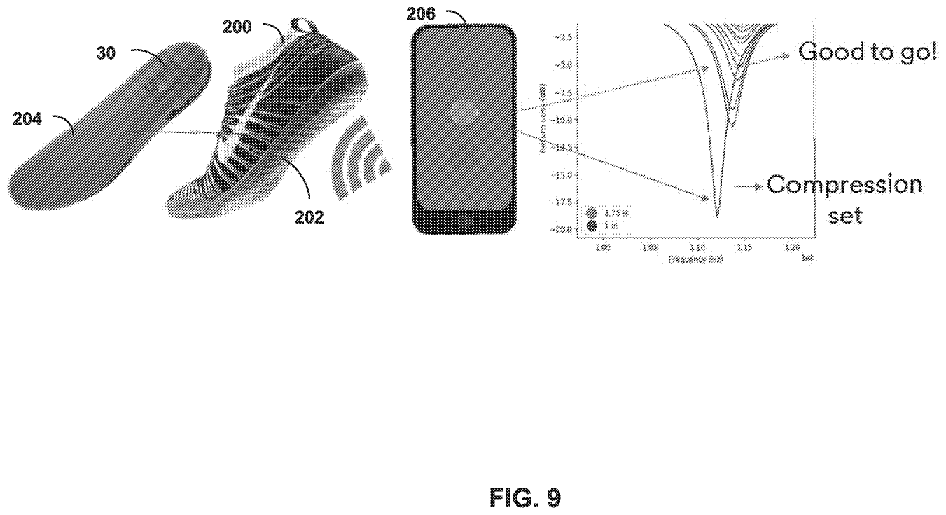

FIG. 9 is a diagram illustrating techniques for determining when the sole of a shoe no longer provides the support needed, in accordance with one aspect of the present disclosure. In the example approach of FIG. 9, a running shoe 200 includes a sole 202 having a thickness that, when the shoe is new, provides a degree of compression to the wearer of the shoe that protects the wearer to a degree from the jarring impact of the shoe to the ground when running. Typically, it is recommended that runners replace their shoes every 3 months or every 300 miles. The replacement interval can, however, vary depending on the individual. Some individuals may need to replace their shoes sooner, and some may wear them longer.

In one example approach, a simple passive SansEC sensor 30 is attached to an insole 204 and the insole 204 is inserted in the shoe 200. An interrogation module 206 is placed against the bottom of sole 202 and distance is measured from the interrogation module 16 to the SansEC sensor 30 by stimulating sensor 30 and receiving its response. The distance measurement may, for instance, be used to calculated how much the sole 202 has compressed, providing a more accurate measurement of wear to the user. In one example approach, the sensor 30 is incorporated into insole 204. In another example approach, the sensor 30 is placed between sole 202 and insole 204.

In one example approach, a machine learning algorithm is trained based on the limited parameters of the shoe application. In some example approaches, interrogation module 16 is a smartphone running an application used to determine wear as described above and having a user interface 40 that displays a stoplight icon with green, yellow or red lights indicating that the shoe 200 is fine, that it is approaching replacement or that it needs to be replaced, respectively.

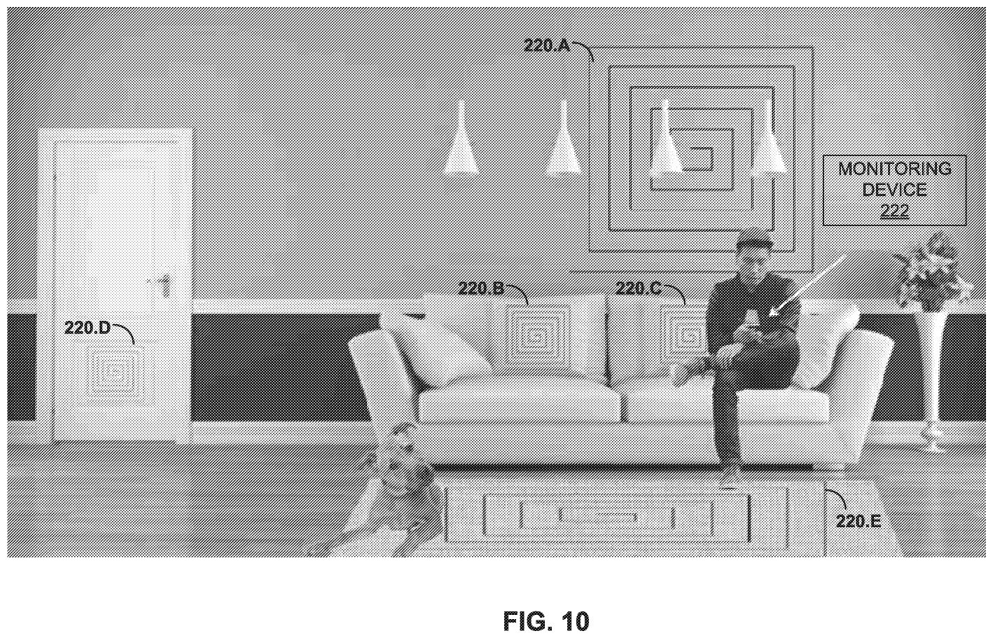

A sensor 30 may be used in a variety of household applications. For instance, water damage from leaking pipes or damaged exteriors in homes can be greatly detrimental to the safety of a home, particularly if the leak is slow and remains hidden in the walls, it could go on for months without notice, leading to severe rot, or the growth of mold within the walls. The costs to repair this kind of damage is often very high. FIG. 10 is a diagram illustrating an example approach to detecting wetness, in accordance with an aspect of this disclosure. As shown in FIG. 10, in one example approach, large scale sensors 220.A-220.E (“large scale sensors 220”) are distributed throughout a room. In the example shown in FIG. 10, a large area sensor 220.A is integrated into drywall or post applied to walls, large scale sensors 220.B and 220.C are integrated into or post applied to cushions of a couch, and a large area sensor 220.D is integrated into or post applied to a door. The working distance of the antennas is dependent on the size, therefore a larger size antenna is able to communicate across enough of a distance to interact with a monitoring device 222 integrated within a smartphone or a smart home device, such as a Nest or a Google Home device available from Google. In order to increase the operational range of the antenna, the frequency may be lowered, which increases the wavelength, and working distances for antennas are typically proportional to half the wavelength. Therefore, larger antennas as depicted in FIG. 10, which may radiate at approximately 10 MHz, may work at distances of up to 15 meters away from a smartphone or a smart home device acting as monitoring device 222.

In another example approach, a large area sensor 220 is integrated into or applied to roof sheathing as a wetness/moisture sensor configured to detect minor water leaks. In another example approach, a large area sensor 220 is placed at the bottom of a garden bed or planter to detect soil moisture level and temperature. In one such example approach, monitoring device 222, in response to one or more of the temperature and moisture level readings, initiates watering of the garden bed or planter via, for example, a sprinkler system or robotic watering system.

In another example approach, as shown in FIG. 10, a large area sensor 220.E is woven into carpet or applied to the underside of carpet as a wetness/moisture sensor. In one such approach, sensor 220.E may be configured to be uses as a spill detector on large area carpets, allowing a homeowner to address unknown spills by receiving phone alerts, or notifying a robotic vacuum that it should start a new cleaning cycle immediately and address the spill.

As can be seen in FIG. 2B, the return loss of the signal in decibels goes from approximately −48 dB to approximately −17 dB as humidity at sensor 220 increases from 33% to 73%. In the household wetness application, sensor 220 exhibits similar behavior as sensor 220 absorbs moisture.

Large area sensors 220 have other applications as well. In one example approach, a sensor 220.E woven into carpet or applied to the underside of carpet may be used, for instance, as a pressure sensor in a home security system, or as a temperature or humidity sensor. Similarly, any of the other sensors 220 may be used, for instance, as a temperature or humidity sensor. In some such example approaches, a smart home device is configured as a monitoring device 222 used to query large area sensor 220. As in the example approach of FIG. 1, the smart home device may include a computing device 14 and an interrogation module 16. Computing device 14 may include one or more processors 20 connected to memory 22.

In some example approaches, monitoring device 222 generates the external oscillating magnetic field and receives a signal from the large area sensor 220 generated in response to the external oscillating magnetic field. In one such example approach, monitoring device 222 includes instructions 26 that when executed by the one or more processors 20 cause the one or more processors to compare data generated from the signal received from the large area sensor 220 to data previously received from the sensor 30 to determine changes in the data and to estimate, based on the changes in the data, changes in one or more of the environmental factors around large area sensor 220. In some such example approaches, data measuring one or more of the other parameters that cause changes in the response of large area sensor 220 is supplied by one or more external devices, or by the monitoring device 222 itself, to make prediction of a desired parameter more accurate. In one such example approach, monitoring device 222 is placed in a permanent location in order to remove the effect of changing distances between the large area sensor 220 and monitoring device 222 from impacting the calculations of the desired parameters.

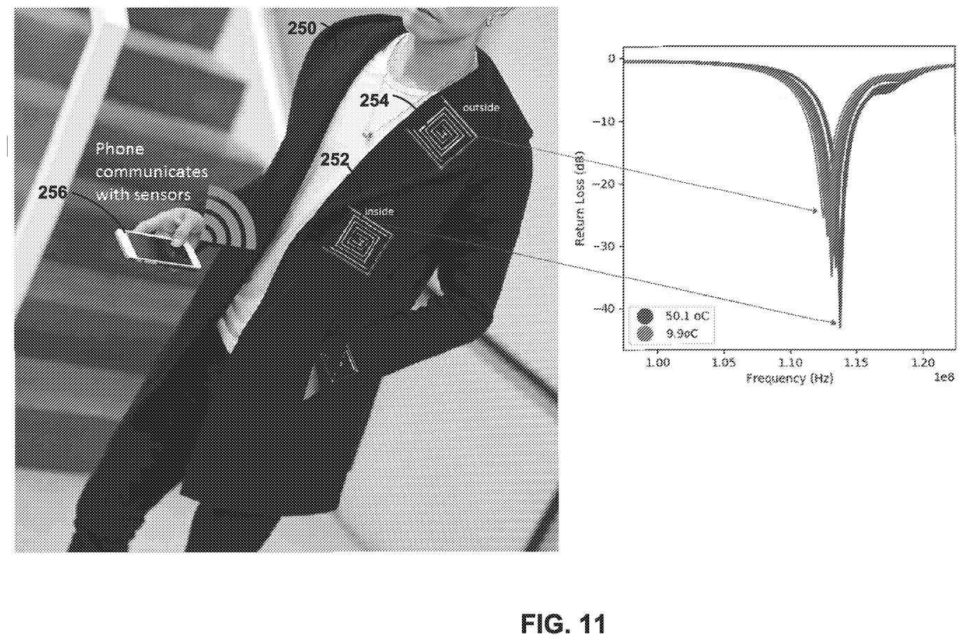

A sensor may be used in an article of clothing. FIG. 11 is an illustration of an article of clothing having an inside sensor and an outside sensor, in accordance with an aspect of this disclosure. In the example shown in FIG. 11, the article of clothing is outerwear. In the example approach of FIG. 11, a jacket 250 includes two or more textile sensors 30, including an inside sensor 252 and an outside sensor 254. In one such example approach, the sensors 252 and 254 are used to determine the temperature inside and outside of the jacket, as well as the humidity. In one example approach, monitoring device 256 is a smart phone or other such device. In one such example approach, monitoring device 256 operates as an interrogation module 16 to determine temperature and humidity and uses the determined temperature and humidity to predict the approximate time the user will have to stay warm outside. This is possible because the insulation has a pre-determined Clo (warmth), which will lose heat based on the flux between the inside and outside of the jacket. For example, the colder it is outside the jacket, the faster the wearer will become cold; that is, the greater the differential between the inside and outside of the jacket, the faster the jacket will lose heat.

The return loss graph of FIG. 2D is replicated in FIG. 11. As can be seen in FIG. 2D and FIG. 11, the return loss of the signal in decibels goes from approximately −42 dB to approximately −17 dB as temperature decreases from approximately 50° C. to 10° C. On a cold day, outer sensor 254 provides resonant frequency signals that approach −17 dB while inner sensor 252 provides resonant frequency signals with higher return loss (approaching −42 dB).

In some example approaches, data measuring one or more of the other parameters that cause changes in the response of sensor 252 or 254 is supplied by one or more external devices, or by the monitoring device 256 itself, to make prediction of temperature more accurate. In some such example approaches, monitoring device 256 is placed against the article of clothing at specific positions in order to remove the effect of the changing distances between monitoring device 256 and sensors 252 and 254 impacting the calculations of the desired parameters. In some such example approaches, the specific positions are marked on the article of clothing.

In one example approach, monitoring device 256 includes a comfort prediction module that operates with sensors 252 and 254 to predict how long the wearer will be comfortable in the current environment. In one such example approach, the comfort prediction module determines a predicted length of time the wearer will be comfortable based on the pre-determined Clo and the responses from sensors 252 and 254. In another such example approach, the comfort prediction module determines a predicted length of time the wearer will be comfortable based on external readings of the outside temperature and humidity, the pre-determined Clo and the responses from sensors 252 and 254.

In yet other example approach, the comfort prediction module determines a predicted length of time the wearer will be comfortable based on one or more of the responses from sensors 252 and 254, physiological characteristics (e.g., heart rate, breathing rate, body temperature, etc.) of the user that is wearing the article of clothing, environmental characteristics (e.g., air temperature, humidity, ambient light, etc.), properties of the article worn by the user (e.g., the pre-determined Clo, the type of material of the article, age of the article, etc.), user information (e.g., historical comfort information, user activity information, etc.), or any combination thereof.

In one example approach, monitoring device 256 may perform one or more operations in response to a prediction that the individual is likely to be uncomfortable, such as adjusting operation of article 250. In some examples, monitoring device 256 automatically adjusts at least one temperature control device (i.e., heating device, cooling device, venting device). For example, monitoring device 256 may automatically activate a heating or cooling device. As another example, monitoring device 256 may automatically output a command to adjust an aperture, such as a zipper or drawstring. For example, monitoring device 256 may output a command to actuate (e.g., open or close) a zipper or adjust (e.g., tighten) a drawstring.

Sensor 30 may be used in other ways with articles of clothing. For instance, an article of clothing may include a sensor 30 used to detect wetness. Such an approach may be used in diapers or in clothing worn over diapers to notify a caregiver of the need to change a diaper.

A sensor may be used in bed linens to regulate temperature in the bed. During REM sleep the body does not regulate temperature, sometimes resulting in people overheating or limbs “falling asleep” without the person's knowledge. Having sensors 30 integrated into the bed sheets, or in a sleep garment, allows one to track personal temperature wirelessly, and to trigger the bed and sheets to heat up or cool down as needed to keep the person at a comfortable temperature.

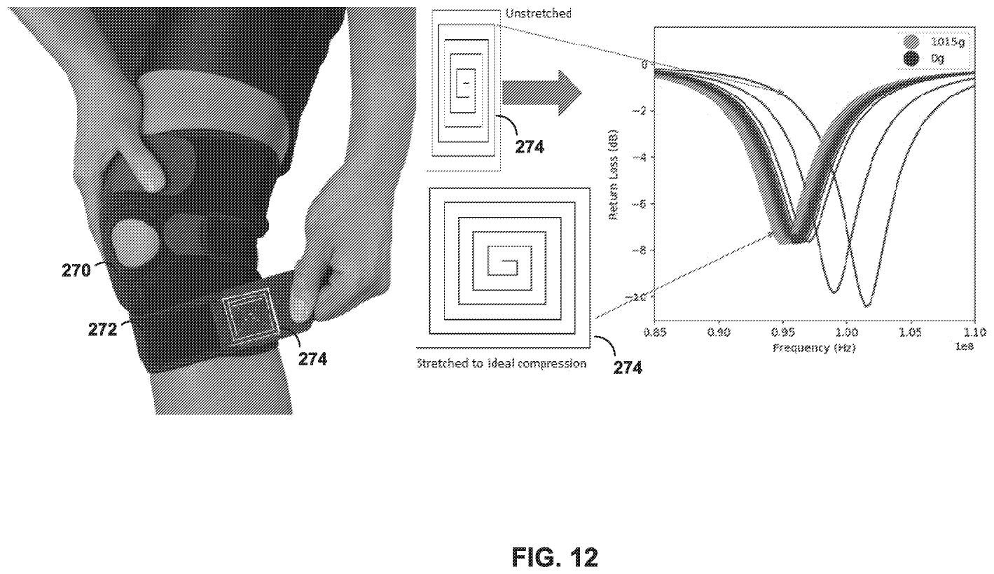

A sensor may be used in a brace to inform the user when an ideal amount of compression has been achieved. FIG. 12 is an illustration showing a brace having an integrated SansEC sensor, in accordance with an aspect of this disclosure. With all SansEC antennas, the shape and size greatly affects the resonant frequency as it is related to the inductance and capacitance of the coil. Therefore, in one example approach, a SansEC sensor 274 is placed (adhered, stitched, knitted, woven) onto an elastic substrate 272 (like a spandex fabric) used in a brace 270. As can be seen in FIG. 12, when not in use, the elastic substrate 272 is unstretched and the SansEC sensor 274 is in a first shape. When, however, the brace is applied to a patient, the sensor 274 in brace 270 is stretched. A monitoring device 12 (e.g., a smartphone) queries the brace and, if the brace is stretched correctly, the device 12 indicates to the user that the brace is on correctly.

The return loss graph of FIG. 2C is replicated in FIG. 12. As can be seen in FIG. 2C and FIG. 12, the return loss of the signal in decibels goes from approximately −12 dB to approximately −8 dB as pressure on sensor 274 decreases. In the brace application, sensor 274 exhibits similar behavior as sensor 274 is stretched to approximately ideal compression.

Over time the elastic in the brace can wear out. In one example approach, if the brace were stretched out beyond a desired amount, monitoring device 12 detects the resulting deformation in sensor 274, and tells the user to replace their brace 270.

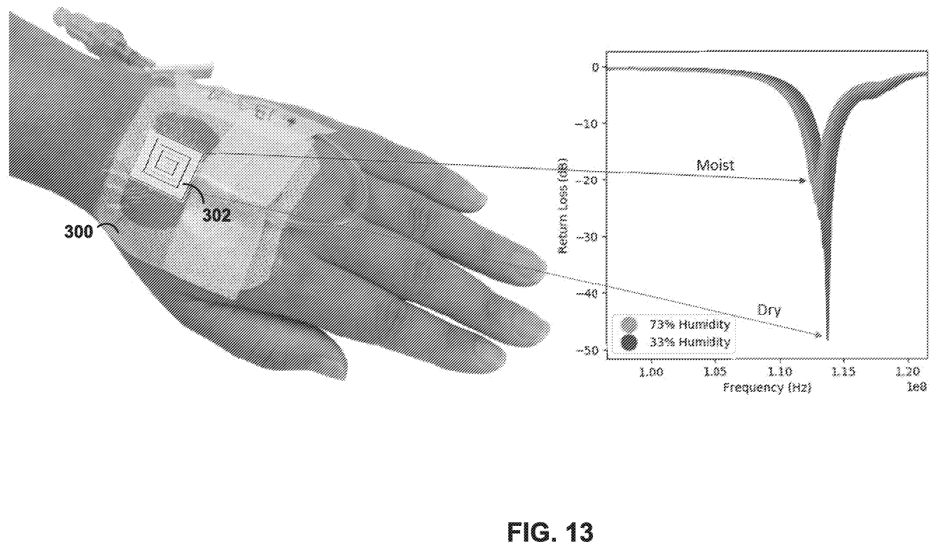

A sensor may be used in medical applications. FIG. 13 is an illustration showing a bandage 300 having an integrated SansEC sensor 302, in accordance with an aspect of this disclosure. In one example approach, bandage 300 includes a bandage substrate; in one such example approach, a SansEC sensor 302 is printed onto the bandage substrate (such as a Nexcare Tegaderm substrate) and used to determine the level of moisture in the wound area as well as the skin temperature. A monitoring device 12 uses the determined parameters to notify a user or caregiver when the bandage should be changed. Such an approach is especially important in applications for patients who need regular bandage changing, or who may not be regularly supervised by a medical professional or caregiver.

In some example approaches, bandage 300 is an adhesive article suitable for application to skin. Therefore, bandage 300 may be a medical tape, bandage, or wound dressing. In some example approaches, bandage 300 may be an IV site dressing, a buccal patch, or a transdermal patch. Bandage 300 may, in some instance, be adhered to the skin of humans and/or animals. In one example approach, bandage 300 includes a bandage substrate, a primer layer disposed on the substrate and a silicone adhesive disposed on the primer layer. In some example approaches, bandage 300 includes other materials such as polymeric materials, plastics, natural macromolecular materials (e.g., collagen, wood, cork, and leather), paper, films, foams, woven cloth and non-woven cloth, and combinations of these materials.

In one example approach, SansEC sensor 302 is integrated into a fabric bandage substrate by, for instance, weaving the sensor 302 into the bandage substrate or by printing the sensor 302 onto the fabric bandage substrate. In another example approach, sensor 302 is woven into or otherwise integrated an absorbent pad attached to the bandage substrate.

The return loss graph of FIG. 2B is replicated in FIG. 13. As can be seen in FIG. 2B and FIG. 13, the return loss of the signal in decibels goes from approximately −48 dB to approximately −17 dB as humidity at sensor 302 increases from 33% to 73%. In the bandage application, sensor 302 exhibits similar behavior as sensor 302 absorbs moisture from the wound.

In one or more examples, the functions described in the context of monitoring devices 12, 206, 222 and 256 may be implemented in hardware, software, firmware, or any combination thereof. If implemented in software, the functions may be stored on or transmitted over, as one or more instructions or code, a computer-readable medium and executed by a hardware-based processing unit. Computer-readable media may include computer-readable storage media, which corresponds to a tangible medium such as data storage media, or communication media including any medium that facilitates transfer of a computer program from one place to another, e.g., according to a communication protocol. In this manner, computer-readable media generally may correspond to (1) tangible computer-readable storage media, which is non-transitory or (2) a communication medium such as a signal or carrier wave. Data storage media may be any available media that can be accessed by one or more computers or one or more processors to retrieve instructions, code and/or data structures for implementation of the techniques described in this disclosure. A computer program product may include a computer-readable medium.

By way of example, and not limitation, such computer-readable storage media can comprise RAM, ROM, EEPROM, CD-ROM or other optical disk storage, magnetic disk storage, or other magnetic storage devices, flash memory, or any other medium that can be used to store desired program code in the form of instructions or data structures and that can be accessed by a computer. Also, any connection is properly termed a computer-readable medium. For example, if instructions are transmitted from a website, server, or other remote source using a coaxial cable, fiber optic cable, twisted pair, digital subscriber line (DSL), or wireless technologies such as infrared, radio, and microwave, then the coaxial cable, fiber optic cable, twisted pair, DSL, or wireless technologies such as infrared, radio, and microwave are included in the definition of medium. It should be understood, however, that computer-readable storage media and data storage media do not include connections, carrier waves, signals, or other transient media, but are instead directed to non-transient, tangible storage media. Disk and disc, as used, includes compact disc (CD), laser disc, optical disc, digital versatile disc (DVD), floppy disk and Blu-ray disc, where disks usually reproduce data magnetically, while discs reproduce data optically with lasers. Combinations of the above should also be included within the scope of computer-readable media.

Instructions may be executed by one or more processors, such as one or more digital signal processors (DSPs), general purpose microprocessors, application specific integrated circuits (ASICs), field programmable logic arrays (FPGAs), or other equivalent integrated or discrete logic circuitry. Accordingly, the term “processor”, as used may refer to any of the foregoing structures or any other structure suitable for implementation of the techniques described. In addition, in some aspects, the functionality described may be provided within dedicated hardware and/or software modules. Also, the techniques may be fully implemented in one or more circuits or logic elements.

The techniques of this disclosure may be implemented in a wide variety of devices or apparatuses, including a wireless handset, an integrated circuit (IC) or a set of ICs (e.g., a chip set). Various components, modules, or units are described in this disclosure to emphasize functional aspects of devices configured to perform the disclosed techniques, but do not necessarily require realization by different hardware units. Rather, as described above, various units may be combined in a hardware unit or provided by a collection of interoperative hardware units, including one or more processors as described above, in conjunction with suitable software and/or firmware.

It is to be recognized that depending on the example, certain acts or events of any of the methods described herein can be performed in a different sequence, may be added, merged, or left out altogether (e.g., not all described acts or events are necessary for the practice of the method). Moreover, in certain examples, acts or events may be performed concurrently, e.g., through multi-threaded processing, interrupt processing, or multiple processors, rather than sequentially.

In some examples, a computer-readable storage medium includes a non-transitory medium. The term “non-transitory” indicates, in some examples, that the storage medium is not embodied in a carrier wave or a propagated signal. In certain examples, a non-transitory storage medium stores data that can, over time, change (e.g., in RAM or cache).

Various examples of the disclosure have been described. These and other examples are within the scope of the following claims.

1. A system comprising:
an article having an open circuit resonator sensor, wherein the sensor includes an approximately planar open circuit pattern of electrically conductive material configured to generate a signal when wirelessly powered by an external oscillating magnetic field, wherein the signal varies as a function of one or more environmental factors associated with an environment around the sensor;
an interrogation module configured to generate the external oscillating magnetic field, to receive the signal generated by the sensor, and to capture data representative of the received signal; and
a computing device coupled to the interrogation module, wherein the computing device comprises a memory and one or more processors coupled to the memory, wherein the memory comprises instructions that when executed by the one or more processors cause one or more of the processors to:
receive the captured data;
compare the captured data to previously captured data; and
estimate, based on the changes in the captured data, changes in one or more of the environmental factors.

2. The system of claim 1, wherein the sensor is a Sans Electrical Connection (SansEC) sensor.

3. The system of claim 1, wherein one or more of the sensor's return loss, the sensor's resonant frequency and the sensor's frequency of peak resistance change in response to changes in one or more of the environmental factors.

4. The system of claim 1, wherein the signal varies as a function of one or more of temperature at the sensor, moisture at the sensor, humidity at the sensor, pressure on the sensor, and distance from the interrogation module to the sensor.

5. The system of claim 1, wherein the article includes fabric and wherein the sensor is textile assembled into the fabric or woven into the fabric.

6. The system of claim 1, wherein the article is a shoe with a compressible sole and wherein the sensor is positioned within the shoe to measure performance of the sole.

7. (canceled)

8. (canceled)

9. The system of claim 1, wherein the article is an article of clothing and the sensor is printed on or within the article of clothing, attached to the article of clothing, or woven into the article of clothing.

10. (canceled)

11. (canceled)

12. The system of claim 1, wherein the article is an article of clothing, wherein the resonator sensor is one of an inner resonator sensor and an outer resonator sensor, wherein the inner resonator sensor is integrated within or attached close to an inner surface of the article of clothing and the outer resonator sensor is integrated within or attached close to an outer surface of the article of clothing, and
wherein the computing device determines a comfort level for a wearer of the article of clothing based on signals received from the inner and outer resonator sensors.

13. (canceled)

14. The system of claim 1, wherein the article is a brace, and
wherein the computing device determines fit of the brace based on the signal received from the resonator sensor.

15. (canceled)

16. The system of claim 1, wherein the article is a bandage and the resonator sensor is integrated into the bandage, and
wherein the computing device detects changes in the bandage based on changes in the signal received from the resonator sensor.

17. The system of claim 1, wherein the electrically conductive material includes one or more of a printed pattern of conductive material, a wire, a conductive yarn, a conductive fiber, a conductively coated textile, a metal, an electrically conductive carbon, and electrically conductive polymers.

18. (canceled)

19. The system of claim 1, wherein the signal changes as a function of one or more of bending the resonator sensor, rotating the interrogation module around an axis orthogonal to the plane of the resonator sensor and changing an angle between the interrogation module and the plane of the resonator sensor.

20. A system comprising:
an article having an open circuit resonator sensor, wherein the resonator sensor includes an approximately planar open circuit pattern of electrically conductive material configured to generate a signal when the resonator sensor is wirelessly powered by an external oscillating magnetic field, wherein the signal varies as a function of one or more environmental factors associated with an environment around the resonator sensor;
an interrogation module configured to generate the external oscillating magnetic field, to receive the signal from the resonator sensor, and to capture data representative of the received signal; and
a machine-learning system coupled to the interrogation module, wherein the machine-learning system applies the captured data to a trained machine-learning model to detect changes in one or more of the environmental factors.

21. The system of claim 20, wherein the open circuit resonator sensor is a Sans Electrical Connection (SansEC) sensor.

22. The system of claim 20, wherein the signal changes as a function of one or more of temperature at the resonator sensor, humidity at the resonator sensor, pressure on the resonator sensor, distance from the interrogation module to the resonator sensor and an angle between the interrogation module and the plane of the resonator sensor.

23. The system of claim 20, wherein the article is one of a shoe, a wall, a door, a piece of furniture, a carpet, a bandage, clothing, outerwear, a brace, an elastic band, and a bandage.

24. A method of detecting changes in an environment of an open circuit resonator sensor, wherein the an open circuit resonator sensor includes an approximately planar open circuit pattern of electrically conductive material configured to generate a signal when the resonator sensor is wirelessly powered by an external oscillating magnetic field, wherein the signal varies as a function of one or more environmental factors associated with the environment around the sensor, the method comprising:
receiving first data representative of the signal generated by the resonator sensor at a first time;
receiving second data representative of the signal generated by the resonator sensor at a second time, wherein the second time is after the first time;
comparing the second data to the first data to determine changes in the second data; and
estimating, based on the changes in the second data, changes in one or more of the environmental factors.

25. The method of claim 24, wherein estimating changes includes estimating changes in one or more of temperature at the resonator sensor, humidity at the resonator sensor, pressure on the resonator sensor, distance from the interrogation module to the resonator sensor and an angle between an interrogation module and the plane of the resonator sensor.

26. The method of claim 24, wherein the open circuit resonator sensor is a Sans Electrical Connection (SansEC) sensor, wherein estimating changes includes applying the second data to a trained machine-learning model to detect changes in one or more of the environmental factors.

27. (canceled)

28. (canceled)

29. (canceled)

30. (canceled)

31. (canceled)
